(19) United States
(12) Patent Application Publication
(10) Pub. No.: US 2016/0302724 A1
(43) Pub. Date: Oct. 20, 2016

(54) Connected smart belt to measure waist size

(71) Applicants: Carine COULM, Paris (FR); Bertrand Duplat, Paris (FR)

(72) Inventors: Carine COULM, Paris (FR); Bertrand Duplat, Paris (FR)

(21) Appl. No.: 14/392,383

(22) PCT Filed: Nov. 18, 2014
(86) PCT No.: PCT/FR2014/002489

§ 371 Date: Jun. 24, 2016

(30) Foreign Application Priority Data
Sep. 19, 2013 (FR) 13 02175

(51) Int. Cl. A61B 5/00; A41F 9/00; A61B 7/00; A61B 5/11; A61B 5/107; A61B 5/0488

(52) U.S. Cl. CPC A61B 5/6831; A61B 5/1072; A61B 5/04884; A61B 7/008; A61B 5/0024; A61B 5/1121; A61B 5/6823; A41F 9/002; A61B 2562/0247; A61B 2562/0219

(57) ABSTRACT

The current invention describes a belt (1), the fashion accessory to hold pants, including a buckle (C) closing on a leather (S), the leather (S) surrounds the hips of the user, characterized by the fact that the buckle (C) is equipped with and electronic board (10) including a battery (B), a main processor (20), and a wireless communication processor, a connection to sensors in the leather (S), the leather (S) is equipped with a length sensor (11) sensing the length of the belt after the closing on the buckle (C) on the leather (S), the electronic board (10) processing length data and sending them to a remote mobile terminal (T), a motor (30) controlled by the main processor (20) allows to loosen or tighten the belt.

### BACKGROUND OF THE INVENTION

(The invention introduces a new connected smart belt to measure waist size.

There is no solution today to provide a real-time measure of a user waist size. The waist size is the measure of the body circumference at the top of the hips. This measure is significant in many well-being or medical applications. Usually this measure is captured, with lowered or removed pants, with a tape measurer looping around the hips to read on the tape the length of the loop. For this measure to work, the belly has to be freed of any mechanical constraints, the user should relax his core muscles and ideally hold his breath. Typically this measure can be cheated by a user, who would insidiously be pulling is stomach to reduce his belly by contracting his core muscles. Taking this measure is cumbersome, with a need to lower's one pant, and depending overall upon the time of the day chosen to take it. This measure is usually a spot one, and does not cover the evolution of the measure over the whole day. In some applications, as for digestive troubles, for instance bloating, for post-surgery follow up, or even for post pregnancy follow up, it would be extremely interesting to have a real-time reading all along the day of the evolution of the user waist size. It would also be useful to get a measure that cannot be forged by contracting one's core muscles or by holding one's breath out. It should also be noted that it is today impossible to get a real time measure while wearing a pant, especially since the belly may pop out more or less above the belt and therefore taints what would be an accurate measurement.

Of course, the fluctuation of measure taken day after day is also a useful and important indicator. To be precise and meaningful, this daily measure should compare similar situations, for instance whether the waist size is measured before or after a meal, or even before or after a sport session.

The invention presents a belt, the fashion accessory used to hold pants, equipped with a series of sensors, whose analysis gives meaningful physiological real time measures. The main object of the invention is to provide a unbiased measure of the genuine waist size of the user.

An object of the invention is to give a measure allowing to compare the waist size of the user day after day.

An object of the invention is to gather relevant measures without disturbing the user in his or her daily life, and having to ask him or her to lower his or her pants to take the measure.

An object of the invention is to get a sensitive reading of physiological activities, currently unavailable in real time, such as breath, digestion, urinary activity and sexual activity.

An object of the invention is to measure physiological fluctuation on tissues and organs: digestive system, reproductive system, muscle and fat tissues, currently undetectable and unmeasurable with existing techniques.

An object of the invention is to propose a real-time alarm, directly on the belt or transferred on a mobile phone warning the user when a threshold has be reached. A real-time alarm is especially important in applications such as breathing rehabilitation.

In one of its main aspects, the invention takes the shape and size of the traditional belt as a fashion accessory, worn on pants, on a bermuda or on a dress.

In one of its aspects, the invention proposes a belt buckle with an electronic board. The belt buckle is connected to a series of sensors inside the buckle and in the leather. The belt carries a battery, a main processor with memory a communication system (Bluetooth, Wifi or equivalent) as well as embedded sensors. The Belt buckle can also include a inertial sensor, an accelerometer, a gyroscope and an altimeter.

In one of its aspect, the invention proposes that the pressure sensor be positioned over an hard part of the body such as over the hip bones. In other versions, several pressure sensors can be positioned around the belt. If the hip bones are privileged for such sensors, it is not an exclusive position, they can be positioned also directly on the user's belly.

In one aspect, the invention proposes that the belt communicates through wireless communication, such as Bluetooth or Wifi, or any other wireless communication system, with a remote smartphone, or tablet or computer.

In an aspect, the invention proposes to measure the waist size of the user by correlating the length data captured by the length sensor and other data provided by the pressure sensors. By crossing these measures with appropriate formulas, the processor can measure the current waist size of the user in real time.

In an aspect, the invention proposes a length sensor which is a potentiometer included within the belt leather. Once the buckle is closed, the user closes the potentiometer circuit at the buckle-leather connection. The potentiometer then provides the length data to the embedded electronic board.

In an aspect, the belt buckle is equipped of a motor adapting the belt tension and length according to various parameters related to the belt usage.

### BRIEF DESCRIPTION OF THE SEVERAL VIEWS OF THE DRAWINGS

The invention will be better understood looking at the following figures:

FIG. 1 presents the belt principle

### DETAILED DESCRIPTION OF THE INVENTION

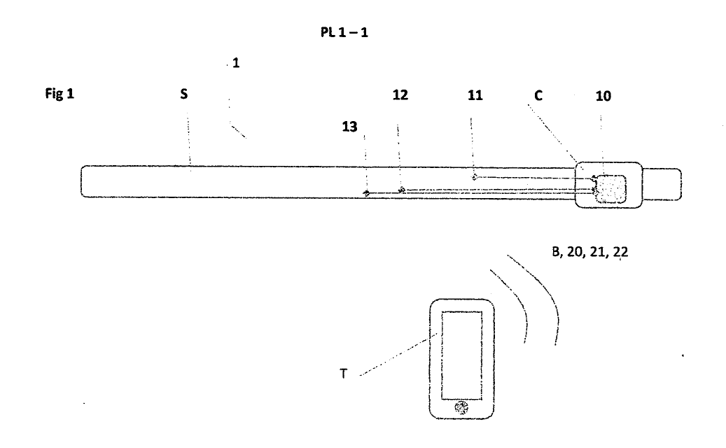

FIG. 1 presents a schematic of the current belt (1) which is first a traditional belt with a leather (S) which closes around the waist with a buckle (C). The buckle is equipped with an electronic board (10) which includes a power source such as a battery (B), a processor and memory chip (20), and a wireless communication chip (21) such as a bluetooth enabled chip, as well as a motion analysis sensor (22) such as an inertial central or an accelerometer or a gyroscope (22). The electronic board can also be connected to sensors inside the belt leather (S).

The motion sensor (22) provides very useful data on the angle that the buckle makes with the horizontal, which is particularly useful for detection of the so called metabolic syndrome.

On the leather (S) at the hip level is a pressure sensor (12) capturing the pressure exerted by the leather on the hip. On the leather (S) a length sensor (11) is embedded. Ideally, this sensor is a potentiometer mounted from the buckle's end to a bit further than the closing distance where the buckle closes on the leather. When the Buckle (C) is closed on the leather (S), the length sensor (11) senses the length up to the closing position and allows to calculate the overall circumference.

The cross analysis of the pressure sensor (12) and the length sensor (11) provides an accurate estimate of the current waist size of the user.

The invention can also include other sensors such as a sound microphone (13) or a electromyography EMG sensor. Data generated by the sensors are processed by the main processor (20) and then wirelessly transmitted to a mobile terminal (T) which displays meaningful data thanks to a specialized App and can also provide insight to the user and communicate with health professionals. A motor (30) allows to automatically regulate the belt tension and length. This motor is connected to the main processor (20) and act according to the main processor commands, and/or those of the smartphone.

The current invention describes a belt, the fashion accessory, including a buckle (c) closing on the leather (S), the leather surrounding the user hips, characterized by the fact that the buckle includes a electronic board (10) itself including a battery (B), a main processor with memory (20), a wireless communication processor (21), a connection to sensors included in the buckle as well as potentially in the leather (S), the leather is also equipped of a length sensor (11) which senses the closed length of the buckle (C) on the leather (S), the electronic board (10) processes length data among others and sends them to a remote mobile terminal (10).

The current invention describes a belt (1) characterized by the fact that the measure of the leather around the user is coupled to a measure of pressure coming from a pressure sensor (12) positioned on the leather (S) to be processed by the main processor (20) to provide an estimate of the current user waist size.

The current invention describes a belt (1) characterized by the fact that the leather includes a sound microphone (13) to listen to the user belly and a EMG sensor.

The current invention present a belt (1) characterized by the fact that a motion analysis sensor (22) is embedded in the electronic board (10) to provide meaningful data such as the angle of the buckle with the vertical. The current invention present a belt (1) characterized by the fact that there is a wireless communication between the belt (C) and a remote mobile terminal (T) of Bluetooth type or any other wireless communication system.

The current invention presents a belt (1) characterized by the fact that a motor (30) is mounted on the buckle (C) or on the leather (S) and automatically regulates the belt (1) tension and length according to the instructions provided by the processor (20), and/or directly by the mobile terminal (T).

Clearly many variations of the current invention are covered without being explicitly described as such, but as various combinations of the different elements in the description. It is easy to see that many variations potentially susceptible to be combined can be added without leaving the invention framework.

1. Belt (1), the fashion accessory, including a buckle (C) closing on a leather (S), the leather (S) surrounding the hips of the user, the buckle (C) is equipped with and electronic board (10) including a battery (B), a main processor with memory (20), and a wireless communication processor (21), a connection to sensors in the buckle and the leather (S), the leather (S) is equipped of a length sensor (11) sensing the length of the closing of the buckle (C) on the leather (S), the electronic board processing the length data and sending them to a remote mobile terminal (T) characterized by the fact that a connected motor (30) is mounted on the buckle (C) or the leather (S) and regulates automatically the belt (1) tension and length according to the instructions coming from the processor (20) depending on the data coming from the different sensors, and/or directly from the mobile terminal (T).

2. Belt (1) according to revendication 1 characterized by the fact that the length data coming for the leather (S) at the user waist is coupled to a pressure sensor (12) positioned on the leather (S) to be processed by the main processor (20) to provide an estimate of the real waist size of the user.

3. Belt (1) according to revendication 1 characterized by the fact that the leather (S) includes a sound sensor (13) listening to the user belly and/or a EMG sensor.

4. Belt (1) according to revendication 1 characterized by the fact that the motion sensor (22) such as an inertial central or an accelerometer (22) is embedded on the electronic board (10) in the buckle, to provide among other things data on the angle between the buckle and the vertical axis.

5. Belt (1) according to revendication 1 characterized by the fact that the wireless connection between the buckle (C) and the remote mobile terminal (T) is Bluetooth or other wireless means
